(19) United States
(12) Patent Application Publication
(10) Pub. No.: US 2001/0052481 A1
(43) Pub. Date: Dec. 20, 2001

(54) INHIBITION OF POPCORN POLYMER GROWTH

(76) Inventors: MAIK ARON, FREINSHEIM (DE); MICHAEL BACHTLER, NEUSTADT (DE); JURGEN KANAND, BAD DURKHEIM (DE)

Correspondence Address: JOHN H SHURTLEFF, 140 S DEARBORN STREET, SUITE 411, CHICAGO, IL, 60603

(21) Appl. No.: 09/260,908

(22) Filed: Mar. 2, 1999

(30) Foreign Application Priority Data
Mar. 5, 1998 (DE) 19809541.4

(51) Int. Cl. C07C 7/20

(52) U.S. Cl. 208/48 AA; 585/950; 208/48 R; 203/8; 203/9

(57) ABSTRACT

Popcorn polymer growth is inhibited in unstabilized materials comprising olefinically unsaturated organic compounds by adding thereto an effective amount of an aliphatic alcohol of the formula I

ROH I

where R is a straight-chain, branched or cyclic C₃-C₂₀-alkyl or alkylene group, the alkylene group bearing a second hydroxyl group.

The present invention relates to a process for the inhibition of popcorn polymer growth in unstabilized materials which comprise olefinically unsaturated organic compounds and are prone to form popcorn polymer.

Many olefinically unsaturated organic monomers, for example styrene and especially dienes having conjugated double bonds, such as 1,3-butadiene or isoprene, are prone to the spontaneous undesirable formation of popcorn polymers, for example during the storage and the transportation of these monomers, their recovery or further processing. These popcorn polymers are usually highly crosslinked, insoluble materials, which form foamy, crusty polymer granules having a cauliflower like structure on the walls of tanks, pipework, apparati and reactors. Popcorn polymerization can result from the action of a variety of factors on the monomer concerned, for example oxygen, heat and rust as well as popcorn polymer particles already present in the monomer, which catalyze popcorn polymer formation.

Popcorn polymer formation is especially critical in the case of conjugated diene monomers, such as 1,3-butadiene or isoprene. Here, popcorn polymerization may be responsible for pipework and reactors becoming plugged and for tank charges polymerizing wholesale and the tanks concerned bursting as a consequence.

To inhibit this popcorn polymerization, monomers which are prone to form popcorn polymer have added to them, even in the course of their being produced, stabilizers which suppress or at least retard the formation of such popcorn polymer in the production, storage, transportation and further processing of these monomers. Monomer from a production facility and commercially available monomer therefore will always include such stabilizers, unless explicitly stated otherwise.

Popcorn polymerization inhibitors used in the case of 1,3-butadiene are customarily radical scavengers, such as 4-tert-butylpyrocatechol (TBC) or 2,6-di-tert-butyl-p-cresol (cf. Ullmann's Encyclopedia of Industrial Chemistry; 5th Ed., Vol. A4, p. 431-446, VCH-Verlagsgesellschaft, Weinheim 1985). As well as these and similar phenols known as radical scavengers, it is prior art to use sodium nitrite (China Synthetic Rubber Industry 11, 357 (1988), sulfur compounds, such as carbon disulfide, hydrogen sulfide, alkanethiols or organic disulfide, alkanethiols or organic disulfides but also elemental phosphorus (U.S. Pat. No. 4,404,413). Similarly, as well as sodium nitrite, the prior art mentions a whole series of other nitrogenous popcorn polymerization inhibitors, for example N,N-dialkylhydroxylamines, trialkylamine oxides (JP-A 223 003 (1988)), nitroso compounds, NO₂, N₂O₃, aromatic amines, hydroxylamines (U.S. Pat. No. 3,148,225), N-hydroxymorpholine (U.S. Pat. No. 3,265,752), N-hydroxypiperidine (U.S. Pat. No. 3,265,751), adducts of phenols and hydroxylamines (U.S. Pat. No. 3,493,603), the products of the reaction of nitrous acid or NO₂ with 1,3-dichlorobut-2-ene or diisobutylene (U.S. Pat. No. 3,560,577), butyraldehyde oxime (U.S. Pat. No. 3,560,577) or the reaction products of dinitrogen tetroxide with diisobutylene (U.S. Pat. No. 3,175,012). U.S. Pat. No. 3,557,232 mentions triarylmethyl chlorides as inhibitors, and WO 92/12948 utilizes alkyl halides as inhibitors for popcorn polymer formation.

Although the aforementioned inhibitors of the prior art do inhibit the formation of popcorn polymers, they prove to be problematical when the stabilized olefinic compound in question is to be further processed by means of homogeneous organometallic catalysts, since these inhibitors can bind to the homogeneous catalysts and thus adversely affect their reactivity and selectivity in relation to the reaction to be catalyzed, or even inactivate these homogeneous catalysts. It is true that most prior art inhibitors are able to suppress the formation of new popcorn polymer seeds, but they are not able to stop popcorn polymerization when popcorn polymers have already been formed in the olefin in question and catalyze popcorn formation most effectively.

JP-A 222 037 (1983) concerns a process for absorption of 1,3-butadiene from gaseous mixtures using, inter alia, butanol as an absorbent. This reference expressly recommends stabilizing the butadiene against popcorn polymerization by addition of TBC to the absorbent.

wo 95/19334 discloses a process where commercial, TBC-stabilized 1,3-butadiene is reacted, inter alia, with alcohols to form the corresponding allyl ethers. The homogeneous or heterogeneous catalysts to be employed for the homogeneous or heterogeneous catalysis of this reaction are over time adversely affected in their reactivity and selectivity, or even inactivated, as a result of reaction with the TBC, which is a powerful complexing agent. Moreover, the sparingly volatile TBC, which is readily soluble in the liquid reaction medium of this process, is carried by the reaction medium into the other parts of the plant, where it has adverse effects on other homogeneously or heterogeneously catalyzed processes. When the unconverted butadiene is separated by distillation from the allyl ether produced therefrom, the sparingly volatile TBC remains in the liquid phase and popcorn polymer will rain down out of the gas phase, making it necessary to shut down and clean out the plant. If TBC is continuously sprayed into the gas space in an attempt to prevent popcorn polymer formation in the gas space of the distillation apparatus, it is true that popcorn polymer formation is prevented, but only at the cost of even more rapid damage to the homogeneous or heterogeneous catalysts, and curtailment of their onstream time, in the subsequent process steps as a consequence of the increased TBC concentration.

It is an object of the present invention to provide a process for inhibiting popcorn polymer growth, to be understood as meaning as including the formation of new popcorn polymer, without the disadvantages of the prior art. More particularly, the inhibitors used for this purpose shall also be capable of inhibiting popcorn formation catalyzed by popcorn polymer, and shall be compatible with a multiplicity of homogeneous organometallic catalysts in the sense of not adversely affecting their catalytic characteristics.

We have found that this object is achieved by a process for the inhibition of popcorn polymer growth in unstabilized material which comprises olefinically unsaturated organic compounds and is prone to form popcorn polymer, which comprises adding to said material an effective amount of an aliphatic alcohol of the formula I

ROH I

where R is a straight-chain, branched or cyclic C₃-C₂₀-alkyl or alkylene group, the alkylene group bearing a second hydroxyl group.

The present invention further provides for the use of aliphatic alcohols of the formula I

ROH I,

where R is a straight-chain, branched or cyclic C₃-C₂₀-alkyl or alkylene group, the alkylene group bearing a second hydroxyl group, for inhibiting popcorn polymer growth in unstabilized materials comprising olefinically unsaturated organic compounds and prone to form popcorn polymer.

The invention further provides compositions comprising material comprising unstabilized butadiene and/or isoprene and from 0.1 to 90% by weight of an alcohol ROH I.

The process of the present invention can be carried out using practically any alcohol ROH I where R is a straight-chain, branched or cyclic alkyl group. Accordingly, the alcohols ROH I can be primary, secondary or even tertiary alcohols. In general, the process of the present invention utilizes C₃-C₂₀-alcohols, preferably C₄-C₁₂-alcohols, particularly preferably C₄-C₆-alcohols. The use of relatively volatile C₄-C₆-alcohols can be beneficial in particular when the olefinic compound to which they are added is further processed in a gas phase reaction where popcorn polymer formation inhibition is required.

There follows a merely exemplary enumeration of suitable alcohols ROH I: propanol, isopropanol, n-butanol, 2-butanol, isobutanol, tert-butanol, n-pentanol, amyl alcohol, neopentyl alcohol, cyclopentyl alcohol, the hexanols, such as n-hexanol or cyclohexanol, the heptanols, octanols, such as n-octanol or 2-ethylhexanol, the nonanols, the decanols, such as n-decanol or 2-propylheptanol, the eicosanols, dodecanols, pentadecanols or octadecanols. Instead of these monoalcohols it is also possible to use the corresponding diols, such as 1,2-propylene glycol, 1,3-propylene glycol, 1,4-butanediol, 1,2-butylene glycol, neopentyl glycol, etc., as alcohols ROH I in the process of the present invention. Preference is generally given to using C₄-C₁₂-alcohols in the process of the present invention, particularly preferably the butanols, especially n-butanol.

The alcohols which are useful in the process of the present invention are effective in inhibiting popcorn polymer growth in the materials comprising olefinically unsaturated organic compounds and prone to form popcorn polymer, not only in the liquid phase but also (self-evidently depending on the volatility of the alcohol in question and on the temperature employed) in the gas phase.

The process of the present invention is useful for inhibiting popcorn polymer growth in all olefinically unsaturated organic compounds prone to form popcorn polymer. Examples of such olefinically unsaturated compounds are styrene and also the dienes with conjugated double bonds which are particularly prone to form popcorn polymer, such as 1,3-butadiene, isoprene or 2,3-dimethylbuta-1,3-diene. The process of the present invention is utilized with particular preference for inhibiting popcorn polymer growth in 1,3-butadiene or isoprene.

For the purposes of this invention, unstabilized materials comprising olefinically unsaturated organic compounds and prone to form popcorn polymers are not only the olefins in question when they contain no stabilizer to inhibit popcorn polymer growth, but also mixtures of these stabilizer-free olefins with other chemical compounds, for example reaction partners, reaction products, impurities, solvents and/or catalysts which do not act as stabilizers for inhibiting popcorn polymer growth. Since it is the object of the present invention to avoid the disadvantages associated with the use of prior art popcorn polymerization inhibitors, the use of materials stabilized against popcorn polymerization in such a way does not form part of the subject-matter of the present invention.

For the purposes of the process of the present invention, the alcohol ROH I can be added to the unstabilized olefinic material prone to popcorn polymerization even in the course of the olefins in question being isolated or else not until the transportation of these olefinic materials, including the transportation to storage facilities, or else only in the course of their storage. If only an olefinic material stabilized with a stabilizer for inhibiting popcorn polymerization is available, the unstabilized olefinic material to be stabilized according to the present invention can be obtained by distillative, extractive, adsorptive or other removal of the added prior art stabilizer and the alcohol ROH I to be used according to the invention added to inhibit popcorn polymer growth. It will be readily understood that instead of an individual alcohol ROH I it is also possible to use mixtures of two or more alcohols ROH I in the process of the present invention.

The addition of the alcohol ROH I to the unstabilized material comprising olefinically unsaturated organic compounds and prone to form popcorn polymer can be effected batchwise or continuously, in which case the addition of the alcohol ROH I is advantageously controlled in such a way that the amount of the alcohol ROH I in this material is never below the level required to inhibit popcorn polymer growth. If, as a consequence of a prolonged period below this level, popcorn polymer is formed, the autocatalytic growth of this popcorn polymer can be inhibited by subsequent metered addition of the alcohol ROH I. Batchwise addition of the alcohol ROH I to the unstabilized olefinic material is generally preferred in the case of tank storage or transportation in closed vessels, whereas to inhibit popcorn polymer growth in flowing unstabilized olefinic material, for example in pipework or reactors, the alcohol ROH I is preferably metered in continuously.

If the material comprising olefinically unsaturated organic compounds and prone to form popcorn polymer is to be reacted or distilled in the gas phase, it can be generally advantageous to inhibit popcorn polymer growth by using an alcohol ROH I which is sufficiently volatile under the conditions employed to inhibit popcorn polymer growth from the gas phase. If the use of such a volatile alcohol ROH I is undesirable, for example for reasons of reaction management or because of separation problems, popcorn polymer growth from the gas phase in such a gas phase reactor or distillation apparatus can advantageously be prevented by passing a less volatile alcohol ROH I into the head or upper part of the gas phase apparatus concerned and dispersing it in the gas space of this apparatus, for example by spraying.

The alcohol ROH I is generally added to the unstabilized material comprising olefinically unsaturated organic compounds and prone to form popcorn polymer in an amount of from 0.1% by weight to 90% by weight.

Even the addition of the small amount of 100 weight ppm of alcohol ROH I is observed to slow down popcorn polymer growth considerably (compared with an unstabilized sample), to an extent which can be sufficient for an effective inhibition of popcorn polymer growth if the material in question is intended for near-term further processing. In the case of prolonged storage or storage under less than ideal conditions, for example in dirty, rusty tanks, it can be advantageous to increase the concentration of the alcohol ROH I in the material in question, similarly when popcorn polymerization has already commenced in a charge of the material in question and catalyzed popcorn polymer growth due to the popcorn polymer already formed is to be inhibited. For the purposes of storage and transportation, the amount of alcohol ROH I added as popcorn polymer growth inhibitor to the unstabilized material comprising olefinically unsaturated organic compounds and prone to form popcorn polymer is generally within the range from 0.1% by weight to 90% by weight, preferably within the range from 0.5% by weight to 25% by weight, particularly preferably within the range from 1 to 15% by weight. It will be appreciated that larger amounts of alcohol ROH I than specified above can be added to the material in question.

If the unstabilized material comprising olefinically unsaturated organic compounds and prone to form popcorn polymer is to be further processed by reacting it with an alcohol ROH I, then the alcohol ROH I added as the reaction partner will also inhibit popcorn polymer growth of the olefinic material in the reactor. In such a case, the amount of alcohol ROH I added, as reaction partner as well as popcorn polymer growth inhibitor, can be equal to the amount of olefinic material, or even exceed it, for example in the case of using a stoichiometric excess of alcohol ROH I, in accordance with the reaction conditions chosen. When the olefin used is already sufficiently stabilized with a popcorn polymerization inhibitor, then the addition of an alcohol ROH I is generally not observed to bring about additional stabilization of the olefin with regard to popcorn polymerization. Since the use of an alcohol ROH I for inhibiting popcorn polymer growth is intended by the present invention to avoid the disadvantages of existing popcorn polymer formation inhibitors, the use of an alcohol ROH I in connection with a prior art inhibitor for stabilizing olefinic materials to avoid popcorn polymer formation does not form part of the subject-matter of the present invention.

As already mentioned, the process of the present invention is particularly advantageous for inhibiting popcorn polymer growth in unstabilized materials comprising olefinically unsaturated organic compounds and prone to form popcorn polymer in the course of their storage and transportation, the process of the present invention being preferably employed for inhibiting popcorn polymer growth in materials comprising conjugated dienes, such as 1,3-butadiene or isoprene or mixtures thereof, especially 1,3-butadiene. Suitable compositions stabilized with alcohols ROH I according to the present invention to inhibit popcorn polymer growth during the storage and transportation generally consist of unstabilized 1,3-butadiene and/or isoprene, including customary synthesis-based impurities, and from 0.1% by weight to 90% by weight, preferably from 0.5% by weight to 25% by weight, particularly preferably from 1% by weight to 15% by weight, of one or more alcohols ROH I, preferably n-butanol.

The process of the present invention is particularly advantageous for inhibiting popcorn polymer growth in, for example, the process for preparing butyraldehyde and/or n-butanol as described in WO 95/19334. When the commercially available, TBC-stabilized 1,3-butadiene used in the process of WO 95/19334 is replaced with 1,3-butadiene stabilized according to the invention with an alcohol ROH I, especially n-butanol, this measure has an advantageous effect on the yield, selectivity and catalyst onstream time of the homogeneously or heterogeneously catalyzed operations utilized in this process.

### EXAMPLES 1 TO 13

Unstabilized 1,3-butadiene in a glass autoclave was admixed with arying amounts of n-butanol and held at 65° C. for 2 months in the resence of an initiator of popcorn polymerization, rusty nails in the case of samples 1 to 7 and popcorn polymer seeds formed during the storage of unstabilized 1,3-butadiene in the case of samples 8 to 13. Samples 1 to 8, hereinafter referred to as blank samples, had no n-butanol added to them.

The amount of n-butanol added to the individual samples is specified in the following table:

| Sample | n-Butanol content | | Seed |
|---|---|---|---|
| 1 | 0 | | Nail |
| 2 | 100 | weight ppm | Nail |
| 3 | 1000 | weight ppm | Nail |
| 4 | 5000 | weight ppm | Nail |
| 5 | 1% | by weight | Nail |
| 6 | 5% | by weight | Nail |
| 7 | 10% | by weight | Nail |
| 8 | 0 | | Popcorn |
| 9 | 1000 | weight ppm | Popcorn |
| 10 | 1% | by weight | Popcorn |
| 11 | 5% | by weight | Popcorn |
| 12 | 10% | by weight | Popcorn |
| 13 | 15% | by weight | Popcorn |

Results:

The two blank samples were full of popcorn polymer after just 4 weeks. Tests 1 and 8 were therefore discontinued for safety reasons.

The sample with 100 weight ppm of n-butanol showed incipient popcorn polymer growth after 2 months. The samples with 1000 and 5000 weight ppm of n-butanol, respectively, were free from popcorn polymer after 2 months, although the samples had become viscous as a result of the formation of butadiene oligomer. The samples with 1% by weight of n-butanol or more were still free-flowing liquids after 2 months without any sign of either popcorn polymer formation or of oligomer formation.

### EXAMPLE 14

Unstabilized 1,3-butadiene in a glass autoclave was admixed either with 5% by weight of n-butanol or with 5% by weight of n-octanol and held at 65° C. for 1 month in the presence of popcorn polymer as initiator of popcorn polymerization. The same test was carried out with a blank sample, to which no alcohol had been added. After 1 month, the samples with alcohol were observed to be free from popcorn polymer formation, whereas the blank sample was found to have formed popcorn polymer to an appreciable extent. The samples with added alcohol were observed in the liquid phase to have formed small amounts of a clear, gellike polymer which was unlike rubbery popcorn polymer and which did not have the adverse properties of popcorn polymer either.

We claim:

1. A process for the inhibition of popcorn polymer growth in unstabilized material which comprises olefinically unsaturated organic compounds and is prone to form popcorn polymer, which comprises adding to said material an effective amount of an aliphatic alcohol of the formula I
ROH I
where R is a straight-chain, branched or cyclic C₃-C₂₀-alkyl or alkylene group, the alkylene group bearing a second hydroxyl group.

2. A process as claimed in claim 1, wherein said olefinically unsaturated organic compound prone to form popcorn polymer contains a conjugated diene group.

3. A process as claimed in either of claims 1 and 2, wherein said olefinically unsaturated organic compound prone to form popcorn polymer is 1,3-butadiene.

4. A process as claimed in either of claims 1 and 2, wherein said olefinically unsaturated compound prone to form popcorn polymer is isoprene.

5. A process as claimed in any of claims 1 to 4, wherein said aliphatic alcohol ROH I is n-butanol.

6. A process as claimed in any of claims 1 to 5, wherein said alcohol ROH I is added to said organic material in an amount of from 0.1% by weight to 90% by weight.

7. A method of using aliphatic alcohols of the formula I
ROH I
where R is a straight-chain, branched or cyclic C₃-C₂₀-alkyl or alkylene group, the alkylene group bearing a second hydroxyl group, for inhibiting popcorn polymer growth in unstabilized materials comprising olefinically unsaturated organic compounds and prone to form popcorn polymer.

8. The method of claim 7, whereunder n-butanol is used for inhibiting popcorn polymer growth in materials comprising 1,3-butadiene or isoprene.

9. Compositions comprising material comprising unstabilized butadiene and/or isoprene and from 0.1 to 90% by weight of an alcohol ROH I as set forth in claim 1.
